(19) United States
(12) Patent Application Publication
(10) Pub. No.: US 2005/0228351 A1
(43) Pub. Date: Oct. 13, 2005

(54) Use of metallic gluconate salts in the production of antimicrobially active substrates

(76) Inventors: Bruno Bret, Wintzenheim (FR); Yves Roussin-Moynier, Wintzenheim (FR); Michel Bourgeois, Lyon (FR); Xavier Normand, Montrottier (FR)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/510,989

(22) PCT Filed: Apr. 15, 2003
(86) PCT No.: PCT/FR03/01194

§ 371 Date: Oct. 13, 2004

(51) Int. Cl. A61F 13/15

(52) U.S. Cl. 604/374

(57) ABSTRACT

The invention relates to the use of metal gluconate salts as antimicrobial agents for the manufacture of substrates based on fibres, especially cellulosic fibres. The invention further relates to substrates based on fibres, especially cellulosic fibres, comprising said metal salts as antimicrobial agents.

The invention relates to the use of particular metal salts for the manufacture of substrates which are based on fibres, especially cellulosic fibres and have antimicrobial activity, especially antibacterial and antifungal activity.

The invention is applied especially in the health, hygiene and food sectors.

Patent EP-B-113 254 describes a nonwoven comprising a web of textile fibres, a polymer-based binder for binding these fibres together, and a small amount of an antimicrobial agent incorporated in this binder, said antimicrobial agent advantageously being selected from halogenated aromatic nitrites, imazalil sulfate, 3,5,3′,4′-tetrachlorosalicylanilide and hexachlorophene.

Patent EP-B-431 002 describes a woven fabric for disinfection or bleaching which comprises a first and a second layer of substrate bonded together with an adhesive polymer and retaining solid particles between them, said particles comprising an agent that releases chlorine.

Patent application WO-A-01 32138 relates to the use of an antimicrobial agent for the manufacture of a disposable wiping article for reducing the number of microbes transferred to the hand when a surface is wiped with said article. The antimicrobial agent is selected from phenolic compounds, isothiazolinone, pyrazole or quaternary ammonium compounds, oxidizing agents, quinolines, guanidines and aldehydes.

Furthermore, the properties of zinc gluconate, copper gluconate and silver gluconate as antiseptics or as sources of supply or supplements are known.

It has now been found, unexpectedly, that substrates comprising certain metal gluconate salts possess antimicrobial activity; it is this finding which forms the basis of the present invention.

Thus, according to a first feature, the invention relates to the use of zinc, silver or copper gluconate as an antimicrobial agent, especially an antibacterial and antifungal agent, for the manufacture of substrates based on fibres, especially cellulosic fibres. The preferred gluconate salt according to the invention is zinc gluconate.

“Substrate based on fibres, especially cellulosic fibres” is understood in terms of the present invention as meaning a substrate consisting partly of cellulosic fibres and more precisely of at least 50% by weight, preferably at least 80% by weight, of cellulosic fibres, which can optionally be mixed with synthetic fibres. In the case of a mixture, the synthetic fiber content of the substrate can be from about 5 to about 40% by weight.

The substrates according to the invention comprise particularly nonwovens based on paper fibres, obtained by the dry method, and pulp wadding based on paper fibres, obtained by the wet method, the latter also being called “tissue paper”.

“Tissue paper” is understood in terms of the present invention as meaning products manufactured from dry and lightweight creped or non-creped paper, such as toilet paper, handkerchiefs, hand wipes, diapers, absorbent papers and serviettes.

Nonwovens are sheets or webs of fibres orientated in one direction or randomly and bonded by mechanical (frictional) means, chemical means (application of adhesive) or thermal means.

As is well known to those skilled in the art, the process for the production of nonwovens based on paper fibres by the dry method consists in treating paper pulp in order to defibrate it dry, forming a voile on a forming cloth, where the individualized fibres are randomly distributed by aeraulics, applying a thermoplastic binder to penetrate the voile formed in this way and enable the fibres to bind together, and then drying and crosslinking the product. The thermoplastic binder can consist of latex, for example an ethylene/vinyl acetate copolymer (EVA), or thermally binding fibres. A sheet of nonwoven obtained by this process generally has a weight of about 40 to 120 g/m².

As is well known to those skilled in the art, the process for the production of pulp wadding based on paper fibres by the wet method consists in depositing an aqueous suspension of paper fibres on a cloth to form a sheet, draining the sheet and then transferring it to a felt by means of which it can be applied with a press against a drying cylinder, where it is dried. The sheet is then detached from the drying cylinder and creped by means of a doctor blade, and then spooled to await conversion to the finished product. The bonding between the paper fibres is effected by means of hydrogen bonds during the wet phase of sheet manufacture.

The conversion phase consists e.g. in assembling several sheets or plies of pulp wadding by calendering, pressure forming and, if appropriate, sizing to give absorbent paper products with a weight ranging from about 8 to 60 g/m².

According to the invention, the substrate comprises an antimicrobial agent, especially an antibacterial and antifungal agent, as defined above.

Thus, according to a second feature, the invention relates to a substrate based on fibres, especially cellulosic fibres, comprising zinc, silver or copper gluconate as an antimicrobial agent, zinc gluconate being preferred.

The antimicrobial agent can be incorporated into the substrate e.g. by spraying a liquid mixture of thermoplastic binder+antimicrobial agent onto the substrate or by impregnating or coating the substrate with the aforementioned mixture, these techniques being well known to those skilled in the art. When the spraying technique is employed, the amount of mixture sprayed onto the substrate is generally between about 12 and 24 g/m².

The concentration of antimicrobial agent in the finished product is about 0.01 to 10% by weight, preferably about 0.05 to 1% by weight. This corresponds to a solids concentration of antimicrobial agent of about 0.006 to 6 g/m², preferably of about 0.03 to 0.6 g/m².

The substrate according to the invention has the following advantages:
it possesses a broad spectrum of activity against Gram-negative microorganisms (for example Pseudomonas aeruginosa) and Gram-positive microorganisms (for example Staphylococcus aureus); and
it can be used safely with food.

The substrate according to the invention, comprising a metal gluconate salt as an antimicrobial agent, can therefore be applied especially:
in sanitary articles such as hand wipes, toilet paper, handkerchiefs, impregnated diapers and absorbent paper;
in feminine hygiene articles, for example as a component (absorbent pad) of sanitary towels, or for babies as an impregnated diaper; and
in food packaging as absorbent paper for meat trays.

The invention will be illustrated with the aid of the Examples and tests which follow. The following abbreviations are used in these Examples and tests:
AN=strain Aspergillus niger ATCC 16404
CA=strain Candida albicans ATCC 10231
EC=strain Escherichia coli ATCC 11229
PA=strain Pseudomonas aeruginosa ATCC 9027
SA=strain Staphylococcus aureus ATCC 6538
(ATCC=American Type Culture Collection)
MIC=minimum inhibitory concentration
EVA=ethylene/vinyl acetate copolymer
CFU=colony forming unit
IZ=inhibition zone

The antimicrobial activity of the substrates according to the invention is evaluated qualitatively and quantitatively according to the standards explained in detail below.

Qualitative Evaluation

a) Swiss standard SNV 195 920: Fabrics—Control of the antibacterial activity: Diffusion Test in Agar

Test-pieces of substrate with a diameter of 25 to 30 mm, treated with the antimicrobial agent according to the invention, are placed on a double layer of nutrient agar inoculated with the test bacteria, and the whole is incubated for 18 h/24 h at 37° C.

The inhibition zone around the test-piece is then measured and is calculated by dividing by 2 the difference between the total diameter of the test-piece plus the inhibition zone, and the diameter of the test-piece.

The test-piece is removed from the contact zone and observed by assessing the bacterial development, making it possible to differentiate between several levels of efficacy.

The strains used in this test are as follows:
Staphylococcus aureus ATCC 6538
Escherichia coli ATCC 11229
Pseudomonas aeruginosa ATCC 9027

b) Swiss standard SNV 195 921: Fabrics—Control of the antifungal activity: Diffusion test in agar

Test-pieces of treated substrate with a diameter of 25 to 30 mm are placed on a double layer of nutrient agar inoculated with the test bacteria, and the whole is incubated.

The inhibition zone around the test-piece is then measured and is calculated by dividing by 2 the difference between the total diameter of the test-piece plus the inhibition zone, and the diameter of the test-piece.

The test-piece is removed from the contact zone and observed by assessing the bacterial development, making it possible to differentiate between several levels of efficacy.

The strains used in this test are as follows:
Aspergillus niger ATCC 16404
Candida albicans ATCC 10231

Quantitative Evaluation

Standard AFNOR XPG 39010: Properties of fabrics—Fabrics and polymer surfaces with antibacterial properties—Characterization and measurement of the bacterio-static activity (inoculation of the test-pieces by transfer)

This standard makes it possible to determine the bacteriostatic activity on fabric and polymer surfaces acting by contact or by diffusion of the antibacterial ingredient, whether the fabrics be hydrophilic or hydrophobic.

The test is performed without maintenance (single use) or after a maintenance cycle.

The samples are washed to remove traces of size and give a hygienically clean product.

The test-pieces are placed on the surface of agar in a Petri dish which has been inoculated by flooding with 1 ml of a bacterial suspension containing 1 to 3.10⁶ CFU/ml.

Substrate-agar contact is assured by applying a 200 g stainless steel cylinder for 1 minute.

The test-piece is placed in a sterile Petri dish, with the inoculated face upwards, and the whole is incubated at 37° C. in a moist chamber for 24 hours or one week.

The test-piece is placed in a sterile sachet. 20 ml of diluent containing a neutralizer are added. The whole is processed in a Stomacher for 1 minute on each side.

This procedure is also applied to untreated cotton test-pieces (used for reference).

Expression of the Results

The bacterial concentrations are expressed as:
CFU (colony forming units)
log CFU
difference of log CFU: Δ₂₄ₕ=log(CFU₂₄ₕ)−log(CFU₀ₕ)
   Δ_{1wk}=log(CFU_{1wk})−log(CFU₀ₕ)

The condition for a substrate to be bacteriostatic according to standard XPG 39010 is as follows:
−2<Δ₂₄ₕ<+2
−2<Δ_{1wk}<+2

The antimicrobial efficacy is superior in the majority of the Examples given below.

The more Δ₂₄ₕ or Δ_{1wk} falls below +2 or falls below −2, the greater is the number of bacteria killed on the substrate by the antimicrobial agent, and the more bactericidal is the substrate.

If the number of CFU is close to zero or equal to zero, the substrate is bactericidal.

### EXAMPLE 1

### Preparation of a Non-Woven Substrate

A solution containing 0.2 g of zinc gluconate, 9.8 g of EVA and 9.8 g of water is prepared. This solution is sprayed (12 g/m²) onto the inner face of a nonwoven weighing 120 g/m² which has been separated into two. This nonwoven is based on exclusively paper fibres and is obtained by the dry method using EVA as the binder. The concentration of zinc gluconate in the finished product is 0.2% by weight.

### EXAMPLE 2

### Preparation of a Non-Woven Substrate

The procedure of Example 1 is repeated except that the nonwoven used, based on exclusively paper fibres and obtained by the dry method using EVA as the binder, has been impregnated with 300% of a standard lotion for diapers prior to the spraying step.

### EXAMPLE 3

### Preparation of a Non-Woven Substrate

The procedure of Example 1 is repeated except that a nonwoven weighing 120 g/m² separated into two and treated on one face with EVA is used. This nonwoven is based on exclusively paper fibres and is obtained by the dry method using EVA as the binder. The solution of zinc gluconate and EVA described in Example 1 is sprayed onto the untreated face of the nonwoven.

### EXAMPLE 4

### Preparation of a Non-Woven Substrate

A nonwoven weighing 60 g/m² is treated industrially by being sprayed on both faces with the solution of zinc gluconate and EVA described in Example 1. This nonwoven is based on exclusively paper fibres and is obtained by the dry method using EVA as the binder.

Test 1

Measurement of the MIC of the Zinc Gluconate

The MIC are shown in the Table below.

TABLE 1

| | Strain | | | | |
|---|---|---|---|---|---|
| | SA | PA | EC | CA | AN |
| MIC (ppm) | 5000 | 12500 | 6250 | 3120 | 6250 |

Test 2

Demonstration of the Antibacterial and Antifungal Activity of Substrates According to the Invention

The activities of the substrates of Examples 1 and 2 were tested according to Swiss standards SNV 195 920 and SNV 195 921. The results are shown in the Table below.

TABLE 2

Strain

| | EC | PA | CA | AN |
|---|---|---|---|---|
| Example 1 | IZ = 0 | IZ = 0 | IZ = 0 | IZ = 0 |
| Example 2 | IZ = 0 | IZ = 0 | IZ = 0 | IZ = 0 |

These results show that the zinc gluconate does not migrate. The substrates according to the invention can therefore be applied especially in the food sector, for example as absorbent paper for meat trays.

Test 3

Demonstration of the Antibacterial Activity of a Substrate According to the Invention

The activity of the substrate of Example 1 on the strains Staphylococcus aureus ATCC 6538 , Pseudomonas aeruginosa ATCC 9027 and Escherichia coli ATCC 11229 was tested according to standard AFNOR XPG 39010 using Columbia agar (marketed by Bio-Merieux), comprising 5% by weight of sheep's blood, as the agar. The results are shown in the Tables below.

TABLE 3
(Staphylococcus aureus)

| | | | |
|---|---|---|---|
| Substrate tested | Log(CFU_{0 h}) | Log(CFU_{24 h}) | Δ_{24 h} |

| | | | |
|---|---|---|---|
| Example 1 | 4.80 | 0.00 [symbol] | −4.80 |
| Reference | 4.83 | 8.64 | 3.81 |

[symbol]

When the number of CFU is equal to zero, log(CFU) is arbitrarily equal to 0.

TABLE 4
(Pseudomonas aeruginosa)

| | | | |
|---|---|---|---|
| Substrate tested | Log(CFU_{0 h}) | Log(CFU_{24 h}) | Δ_{24 h} |

| | | | |
|---|---|---|---|
| Example 1 | 5.26 | 0.00 [symbol] | −5.26 |
| Reference | 5.19 | 9.69 | 4.50 |

[symbol]

When the number of CFU is equal to zero, log(CFU) is arbitrarily equal to 0.

TABLE 5
(Escherichia coli)

| | | | |
|---|---|---|---|
| Substrate tested | Log(CFU_{0 h}) | Log(CFU_{24 h}) | Δ_{24 h} |

| | | | |
|---|---|---|---|
| Example 1 | 5.15 | 0.00 [symbol] | −5.15 |
| Reference | 5.06 | 9.41 | 4.35 |

[symbol]

When the number of CFU is equal to zero, log(CFU) is arbitrarily equal to 0.

Test 4

Demonstration of the Antibacterial Activity of a Substrate According to the Invention

The activity of the substrate of Example 3 on the strains Staphylococcus aureus ATCC 6538 and Pseudomonas aeruginosa ATCC 9027 was tested according to standard AFNOR XPG 39010 using Columbia agar, optionally comprising 5% by weight of sheep's blood, as the agar. The results are shown in the Tables below.

TABLE 6
(Staphylococcus aureus, blood agar)

| | | | |
|---|---|---|---|
| Substrate tested | Log(CFU_{0 h}) | Log(CFU_{24 h}) | Δ_{24 h} |

| | | | |
|---|---|---|---|
| Example 3 | 5.11 | 0.00 [symbol] | −5.11 |
| Reference | 5.13 | 8.18 | 3.05 |

[symbol]

When the number of CFU is equal to zero, log(CFU) is arbitrarily equal to 0.

TABLE 7
(Pseudomonas aeruginosa)

| | | | |
|---|---|---|---|
| Substrate tested | Log(CFU_{0 h}) | Log(CFU_{24 h}) | Δ_{24 h} |

| | | | |
|---|---|---|---|
| Example 3 | 5.08 | 0.79 | −4.29 |
| Reference | 4.94 | 9.10 | 4.16 |

TABLE 8
(Pseudomonas aeruginosa, blood agar)

| | | | |
|---|---|---|---|
| Substrate tested | Log(CFU_{0 h}) | Log(CFU_{24 h}) | Δ_{24 h} |

| | | | |
|---|---|---|---|
| Example 3 | 5.09 | 1.62 | −3.47 |
| Reference | 5.02 | 9.64 | 4.62 |

Test 5

Demonstration of the Antibacterial Activity of a Substrate According to the Invention

The activity of the substrate of Example 4 on the strains Staphylococcus aureus ATCC 6538 and Pseudomonas aeruginosa ATCC 9027 was tested according to standard AFNOR XPG 39010 using Columbia agar comprising 5% by weight of sheep's blood (three test-pieces of Example 4 and two test-pieces of the reference were tested). The results are shown in the Tables below.

TABLE 9
(Staphylococcus aureus)

| Incubation time | | | | | | | | | | |
|---|---|---|---|---|---|---|---|---|---|---|
| Substrate | | 0 h | | | | 24 h | | | | |
| tested | Test-piece | CFU | log(CFU_{0 h}) | SD | Mean | CFU | log(CFU_{24 h}) | SD | Mean | Δ_{24 h(mean)} |
| Example 4 | 1 | 9.98 · 10⁴ | 5.00 | 0.07 | 5.07 | 0.00 | 0.00 [symbol] | 1.76 | [symbol] | −5.07 |
| | 2 | 1.34 · 10⁵ | 5.13 | | | 1.12 · 10³ | 3.05 | | | −2.02 |
| | 3 | 1.23 · 10⁵ | 5.09 | | | 0.00 | 0.00 | | | −5.07 |
| Reference | 1 | 8.41 · 10⁴ | 4.93 | 0.12 | 5.01 | 5.24 · 10⁸ | 8.72 | 0.12 | 8.80 | 3.79 |
| | 2 | 1.25 · 10⁵ | 5.10 | | | 7.69 · 10⁸ | 8.89 | | | |

[symbol]

When the number of CFU is equal to zero, log(CFU) is arbitrarily equal to 0. [symbol]
The mean was not calculated because the difference in the extreme values of the logarithms is greater than 1.

TABLE 10
(Staphylococcus aureus)

| Incubation time | | | | | | | | | | |
|---|---|---|---|---|---|---|---|---|---|---|
| Substrate | | 0 h | | | | 1 week | | | | |
| tested | Test-piece | CFU | log(CFU_{0 h}) | SD | Mean | CFU | log(CFU_{1 wk}) | SD | Mean | Δ_{1 wk(mean)} |
| Example 4 | 1 | 9.98 · 10⁴ | 5.00 | 0.07 | 5.07 | 0.00 | 0.00 | 0.00 | 0.00 | −5.07 |
| | 2 | 1.34 · 10⁵ | 5.13 | | | 0.00 | 0.00 [symbol] | | | |
| | 3 | 1.23 · 10⁵ | 5.09 | | | 0.00 | 0.00 | | | |
| Reference | 1 | 8.41 · 10⁴ | 4.93 | 0.12 | 5.01 | 5.95 · 10⁷ | 7.77 | 0.16 | 7.89 | 2.88 |
| | 2 | 1.25 · 10⁵ | 5.10 | | | 1.01 · 10⁸ | 8.01 | | | |

[symbol]

When the number of CFU is equal to zero, log(CFU) is arbitrarily equal to 0.

TABLE 11
(Pseudomonas aeruginosa)

| Incubation time | | | | | | | | | | |
|---|---|---|---|---|---|---|---|---|---|---|
| Substrate | | 0 h | | | | 24 h | | | | |
| tested | Test-piece | CFU | log(CFU_{0 h}) | SD | Mean | CFU | log(CFU_{24 h}) | SD | Mean | Δ_{24 h(mean)} |
| Example 4 | 1 | 1.44 · 10⁵ | 5.16 | 0.01 | 5.16 | 0.00 | 0.00 | 0.00 | 0.00 | −5.16 |
| | 2 | 1.47 · 10⁵ | 5.17 | | | 0.00 | 0.00 [symbol] | | | |
| | 3 | 1.43 · 10⁵ | 5.15 | | | 0.00 | 0.00 | | | |
| Reference | 1 | 1.41 · 10⁵ | 5.15 | 0.08 | 5.10 | 3.84 · 10⁹ | 9.58 | 0.01 | 9.59 | 4.49 |
| | 2 | 1.10 · 10⁵ | 5.04 | | | 3.95 · 10⁹ | 9.60 | | | |

[symbol]

When the number of CFU is equal to zero, log(CFU) is arbitrarily equal to 0.

The results in Tables 3 to 11 show the excellent antibacterial activity of the substrates according to the invention.

1-5. (canceled)

6. A substrate comprising at least 50% by weight of cellulosic fibres, which substrate comprises zinc, silver or copper gluconate incorporated thereto as an antimicrobial agent.

7. A substrate according to claim 6 which is a sanitary article.

8. A substrate according to claim 6 which is a feminine hygiene article or a baby article.

9. A substrate according to claim 6 which is a food packaging article.

10. A substrate according to claim 6 which is a nonwoven based on paper fibres, obtained by the dry method.

11. A substrate according claim 6 which comprises from about 0.01 to 10% by weight, of antimicrobial agent.

12. A substrate according to claim 6 wherein the antimicrobial agent is zinc gluconate.

13. A sanitary article according to claim 7, which is a hand wipe, toilet paper, a handkerchief, an impregnated diaper or absorbent paper.

14. A feminine hygiene article according to claim 8, which is an absorbent pad.

15. A baby article according to claim 8, which is an impregnated diaper.

16. A food packaging article according to claim 9, which is a nonwoven or absorbent paper for meat trays.

17. A substrate according to claim 11, which comprises from about 0.05 to 1% by weight of antimicrobial agent.

18. A susbtrate according to claim 6, wherein the cellulosic fibres are mixed with about 5 to about 40% by weight of synthetic fibres, based on the weight of the substrate.

19. A sheet of nonwoven as defined in claim 10, which has a weight of about 40 to 120 g/m².

20. A substrate according to claim 6 which is a pulp wadding based on paper fibres, obtained by the wet method.
